(19) United States
(12) Patent Application Publication
(10) Pub. No.: US 2026/0253447 A1
(43) Pub. Date: Aug. 27, 2026

(54) IMAGING SUPPORT DEVICE, PERSONAL IDENTIFICATION METHOD, PROGRAM, AND MEDICAL IMAGE CAPTURING SYSTEM

(71) Applicant: FUJIFILM Corporation, Tokyo (JP)

(72) Inventor: Kazunari HONDA, Tokyo (JP)

(73) Assignee: FUJIFILM Corporation, Tokyo (JP)

(21) Appl. No.: 19/543,908

(22) Filed: Feb. 19, 2026

(30) Foreign Application Priority Data
Feb. 21, 2025 (JP) 2025-026681

(51) Int. Cl. G06V 40/16; A61B 5/00; G16H 10/60

(52) U.S. Cl. CPC G06V 40/172; A61B 5/0037; A61B 5/70; G06V 40/168; G16H 10/60

(57) ABSTRACT

Provided are an imaging support device, a personal identification method, a program, and a medical image capturing system that can realize suitable personal identification for a subject who is lying down, using a captured image of the subject. An imaging support device acquires a captured image of a subject who is lying down, the captured image being acquired by imaging the subject using an imaging apparatus that is fixedly disposed in an imaging room in which a medical image capturing apparatus is provided, extracts face information of the subject from the captured image, acquires master face information of the subject, collates the face information with the master face information, and performs personal identification of the subject based on a collation result of the face information and the master face information.

### CROSS-REFERENCE TO RELATED APPLICATION

The present application claims priority under 35 U.S.C § 119(a) to Japanese Patent Application No. 2025-026681 filed on Feb. 21, 2025, which is hereby expressly incorporated by reference, in its entirety, into the present application.

### BACKGROUND OF THE INVENTION

### 1. Field of the Invention

The present disclosure relates to an imaging support device, a personal identification method, a program, and a medical image capturing system.

### 2. Description of the Related Art

In a medical institution such as a hospital, personal identification of a patient is performed in order to prevent a medical examination operator such as a doctor or a medical technician from making a mistake in a medical examination. In a case in which it is confirmed that the patient is a correct patient and the personal identification of the patient is successful, the medical examination is performed on the patient. The medical examination may include capturing of a medical image of the patient.

JP2021-097870A discloses a patient authentication system that performs patient authentication based on a patient reference face image stored in a reference database and an authentication face image acquired by imaging a patient using a device-side imaging unit provided at an entrance of an examination room.

JP2007-185321A discloses an X-ray apparatus that captures an X-ray image of a subject. The apparatus disclosed in JP2007-185321A includes a moving cart on which an X-ray generation device and a television camera are mounted, acquires a face of the subject on a bed captured by the television camera as personal identification information, collates the personal identification information with master information, and determines whether or not the subject to be imaged is the same as a reserved subject.

### SUMMARY OF THE INVENTION

However, in the system disclosed in JP2021-097870A, the patient is imaged by a reception machine, and it is assumed that the patient can walk on his or her own. Therefore, it is difficult to perform personal identification using a captured image of a subject who is lying on a bed of a medical image capturing apparatus, a stretcher, or the like.

In the apparatus disclosed in JP2007-185321A, it is necessary to adjust a position of the moving cart according to a position of a face of the subject on the bed, and it is necessary to adjust the position of the moving cart according to a position of an imaging target part. That is, it is necessary to perform the position adjustment of the moving cart for the subject on the bed a plurality of times, and there is a concern that a workload of an operator will increase.

The present disclosure has been made in view of such circumstances, and an object thereof is to provide an imaging support device, a personal identification method, a program, and a medical image capturing system that can realize suitable personal identification for a subject who is lying down, using a captured image of the subject.

According to a first aspect of the present disclosure, an imaging support device comprising: a processor; and a memory that stores a program to be executed by the processor, in which the processor is configured to acquire a captured image of a subject who is lying down, the captured image being acquired by imaging the subject using an imaging apparatus that is fixedly disposed in an imaging room in which a medical image capturing apparatus is provided, extract first face information of the subject from the captured image, acquire master face information of the subject corresponding to identification information of the subject, collate the first face information with the master face information, and perform personal identification of the subject based on a collation result of the first face information and the master face information.

According to the imaging support device in the first aspect, the first face information of the subject based on the captured image of the subject who is lying down and the master face information of the subject are collated, and the personal identification of the subject based on the collation result is performed. As a result, suitable personal identification for the subject who is lying down is realized using the captured image of the subject who is lying down.

According to a second aspect of the present disclosure, in the imaging support device according to the first aspect, the processor may be configured to notify that the personal identification of the subject is successful in a case in which a similarity between the first face information and the master face information exceeds a predetermined value.

According to a third aspect of the present disclosure, in the imaging support device according to the first or second aspect, the processor may be configured to register the first face information as the master face information.

According to a fourth aspect of the present disclosure, in the imaging support device according to the third aspect, the processor may be configured to register the master face information in association with medical record information of the subject.

According to a fifth aspect of the present disclosure, in the imaging support device according to any one of the first to fourth aspects, the processor may be configured to acquire the master face information for each subject from a master face information registration device in which the master face information for each subject associated with the identification information for each subject is registered.

According to a sixth aspect of the present disclosure, in the imaging support device according to any one of the first to fifth aspects, the processor may be configured to notify information for prompting update of the master face information for which a predetermined period has elapsed from registration.

According to a seventh aspect of the present disclosure, in the imaging support device according to any one of the first to sixth aspects, the processor may be configured to extract a face region of the subject from the captured image of the subject, cut out the extracted face region from the captured image to generate a first face region image of the subject, and apply the first face region image as the first face information.

According to an eighth aspect of the present disclosure, in the imaging support device according to any one of the first to seventh aspects, the processor may be configured to acquire second face information of the subject from an ID card of the subject in a case in which the master face information of the subject is not registered, and apply the acquired second face information as the master face information of the subject.

According to a ninth aspect of the present disclosure, in the imaging support device according to the eighth aspect, the processor may be configured to register the second face information as the master face information of the subject.

According to a tenth aspect of the present disclosure, in the imaging support device according to the eighth or ninth aspect, the processor may be configured to acquire scan data of a face image of the subject, generate a second face region image of the subject from the scan data of the face image of the subject, and apply the second face region image as the second face information.

According to an eleventh aspect of the present disclosure, there is provided a personal identification method performed by a computer that functions as an imaging support device including a processor and a memory that stores a program to be executed by the processor, the method including acquiring a captured image of a subject who is lying down, the captured image being acquired by imaging the subject using an imaging apparatus that is fixedly disposed in an imaging room in which a medical image capturing apparatus is provided, extracting first face information of the subject from the captured image, acquiring master face information of the subject corresponding to identification information of the subject, collating the first face information with the master face information, and performing personal identification of the subject based on a collation result of the first face information and the master face information.

According to the personal identification method according to the eleventh aspect of the present disclosure, the same action and effect as those of the imaging support device according to the first aspect can be obtained. The configuration requirements of the imaging support device according to the second to tenth aspects can be applied as the configuration requirements of the personal identification method according to other aspects.

According to a twelfth aspect of the present disclosure, there is provided a program for causing a computer that functions as an imaging support device including a processor and a memory that stores a program to be executed by the processor to implement functions of acquiring a captured image of a subject who is lying down, the captured image being acquired by imaging the subject using an imaging apparatus that is fixedly disposed in an imaging room in which a medical image capturing apparatus is provided, extracting first face information of the subject from the captured image, acquiring master face information of the subject corresponding to identification information of the subject, collating the first face information with the master face information, and performing personal identification of the subject based on a collation result of the first face information and the master face information.

According to the program according to the twelfth aspect of the present disclosure, the same action and effect as those of the imaging support device according to the first aspect can be obtained. The configuration requirements of the imaging support device according to the second to tenth aspects may be applied to configuration requirements of the program according to other aspects.

According to a thirteenth aspect of the present disclosure, there is provided a medical image capturing system comprising: a medical image capturing apparatus that generates medical image capturing data of a subject, an imaging apparatus that is fixedly disposed in an imaging room in which the medical image capturing apparatus is provided, and an imaging support device that performs personal identification of the subject, in which the imaging support device includes a first processor and a first memory that stores a program to be executed by the first processor, and the first processor is configured to acquire a captured image of the subject who is lying down, which is acquired by imaging the subject using the imaging apparatus, extract first face information of the subject from the captured image, acquire master face information of the subject corresponding to identification information of the subject, collate the first face information with the master face information, and perform personal identification of the subject based on a collation result of the first face information and the master face information.

According to the medical image capturing system according to the thirteenth aspect of the present disclosure, the same action and effect as those of the imaging support device according to the first aspect can be obtained. The configuration requirements of the imaging support device according to the second to tenth aspects can be applied as the configuration requirements of the medical image capturing system according to other aspects.

According to a fourteenth aspect of the present disclosure, in the medical image capturing system according to the thirteenth aspect, any one of a computed tomography imaging apparatus, a magnetic resonance imaging apparatus, or a medical image augmented reality apparatus may be applied as the medical image capturing apparatus.

According to a fifteenth aspect of the present disclosure, in the medical image capturing system according to the thirteenth or fourteenth aspect, the imaging apparatus may be fixedly disposed on a ceiling of the imaging room, fixedly disposed on a wall of the imaging room, or fixedly disposed using a support member that is erected on a floor of the imaging room.

According to a sixteenth aspect of the present disclosure, in the medical image capturing system according to the thirteenth or fourteenth aspect, the imaging apparatus may be fixedly disposed on the medical image capturing apparatus.

According to a seventeenth aspect of the present disclosure, in the medical image capturing system according to any one of the thirteenth to sixteenth aspects, the medical image capturing apparatus includes a second processor and a second memory that stores a program to be executed by the second processor, and the second processor may be configured to detect a position of an imaging part of the subject in the captured image, and perform movement control of a bed on which the subject is placed in the capturing of the medical image for the subject based on the detected position of the imaging part of the subject.

According to the seventeenth aspect of the present disclosure, in the medical image capturing system, the second processor may be configured integrally with the first processor. For example, one processor device may include a first region and a second region, the first region may function as the first processor, and the second region may function as the second processor.

According to the seventeenth aspect of the present disclosure, in the medical image capturing system, the second memory may be configured integrally with the first memory. For example, one memory device may include a first region and a second region, the first region may function as the first memory, and the second region may function as the second memory.

According to the present disclosure, the first face information of the subject based on the captured image of the subject who is lying down and the master face information of the subject are collated, and the personal identification of the subject based on the collation result is performed. As a result, suitable personal identification for the subject who is lying down is realized using the captured image of the subject who is lying down.

### BRIEF DESCRIPTION OF THE DRAWINGS

FIG. 1 is a functional block diagram showing a configuration example of a medical image capturing system according to an embodiment.

FIG. 2 is a perspective view schematically showing a configuration of an X-ray CT apparatus shown in FIG. 1.

FIG. 3 is a functional block diagram showing an electric configuration of the X-ray CT apparatus shown in FIG. 2.

FIG. 4 is a functional block diagram showing an electric configuration of an authentication device according to a first embodiment.

FIG. 5 is a block diagram showing a hardware configuration example of the electric configuration of the authentication device according to the first embodiment.

FIG. 6 is a flowchart showing a procedure of an authentication method according to the first embodiment.

FIG. 7 is a schematic diagram of a master image display screen.

FIG. 8 is a schematic diagram of a camera image.

FIG. 9 is a schematic diagram of a collation result display screen in a case in which a face region image matches a master image.

FIG. 10 is a schematic diagram of a collation result display screen in a case in which the face region image does not match the master image.

FIG. 11 is a schematic diagram of a collation result display screen in a case in which the face region image does not match the master image.

FIG. 12 is a schematic diagram of an authentication result display screen in a case in which the personal authentication is successful.

FIG. 13 is a schematic diagram of an authentication result display screen in a case in which the personal authentication is unsuccessful.

FIG. 14 is a functional block diagram showing an electric configuration of an authentication device according to a second embodiment.

FIG. 15 is a flowchart showing a procedure of an authentication method according to the second embodiment.

FIG. 16 is a schematic diagram of an ID card reading selection screen.

FIG. 17 is a schematic diagram of a master image registration screen.

FIG. 18 is a schematic diagram of a master image update screen applied to the authentication device according to a third embodiment.

### DESCRIPTION OF THE PREFERRED EMBODIMENTS

Hereinafter, a preferred embodiment of the present invention will be described in detail with reference to the accompanying drawings. In the following description and the accompanying drawings, the same constituent elements are denoted by the same reference numerals, and the duplicated description thereof is omitted. In addition, in the following embodiment, in a case where a plurality of constituent elements are described and listed, it can be interpreted that at least one of the plurality of constituent elements is included.

### Overall Configuration of Medical Image Capturing System

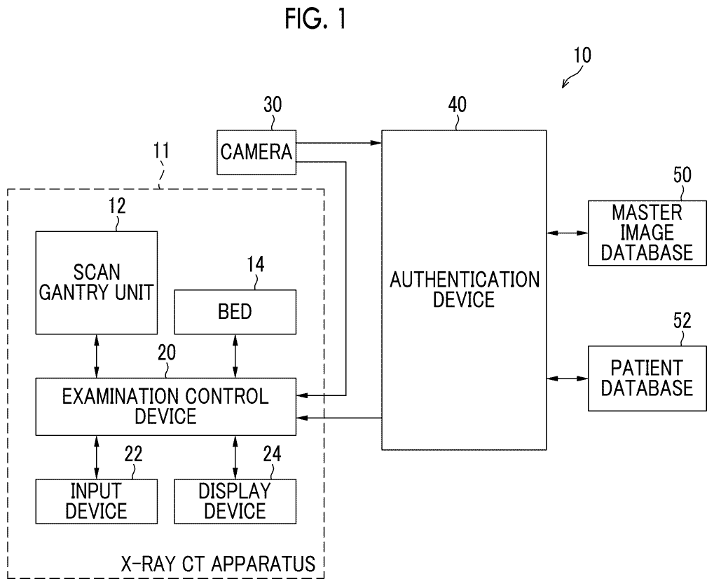

FIG. 1 is a functional block diagram showing a configuration example of a medical image capturing system according to an embodiment. The medical image capturing system 10 comprises an X-ray CT apparatus 11, a camera 30, an authentication device 40, a master image database 50, and a patient database 52.

FIG. 1 shows the X-ray CT apparatus 11 as the medical image capturing apparatus, but the medical image capturing apparatus may be an MRI apparatus or an XR apparatus. It should be noted that CT is an abbreviation for Computed Tomography, MRI is an abbreviation for Magnetic Resonance Imaging, and XR is an abbreviation for eXtended Reality.

The X-ray CT apparatus 11, the camera 30, the authentication device 40, the master image database 50, and the patient database 52 are electrically connected to each other via a network. Examples of the network applied to the medical image capturing system 10 include a LAN. Note that LAN is an abbreviation for local area network.

The X-ray CT apparatus 11 comprises a scan gantry unit 12, a bed 14, and an examination control device 20. An input device 22 and a display device 24 are connected to the examination control device 20. A keyboard, a mouse, a multi-touch panel, other pointing devices, or any combination thereof is applied to the input device 22. The input device 22 may include a code reader that reads a barcode, a two-dimensional code, or the like.

A liquid crystal display, an organic EL display, a projector, or any combination thereof is applied to the display device 24. In a case in which a touch panel display is applied as the display device 24, an operation button or the like can be realized using the touch panel display.

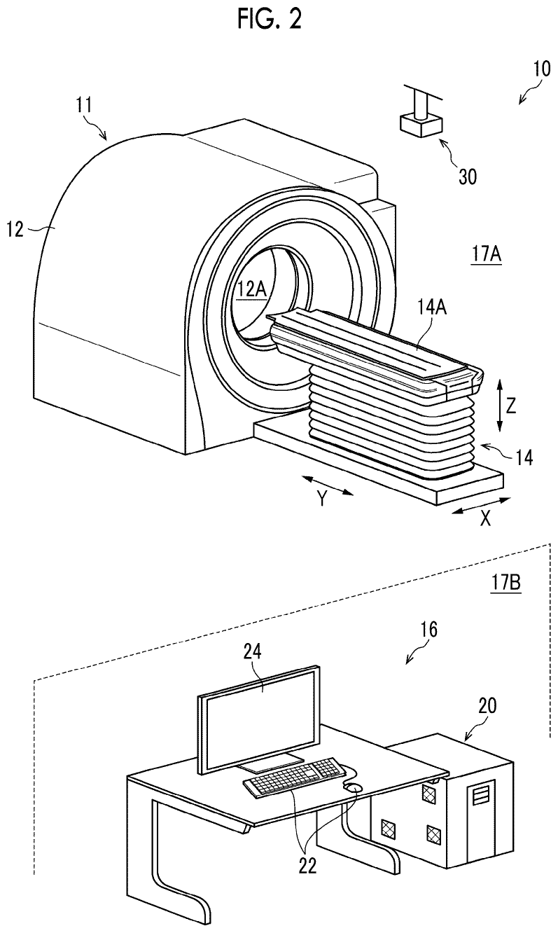

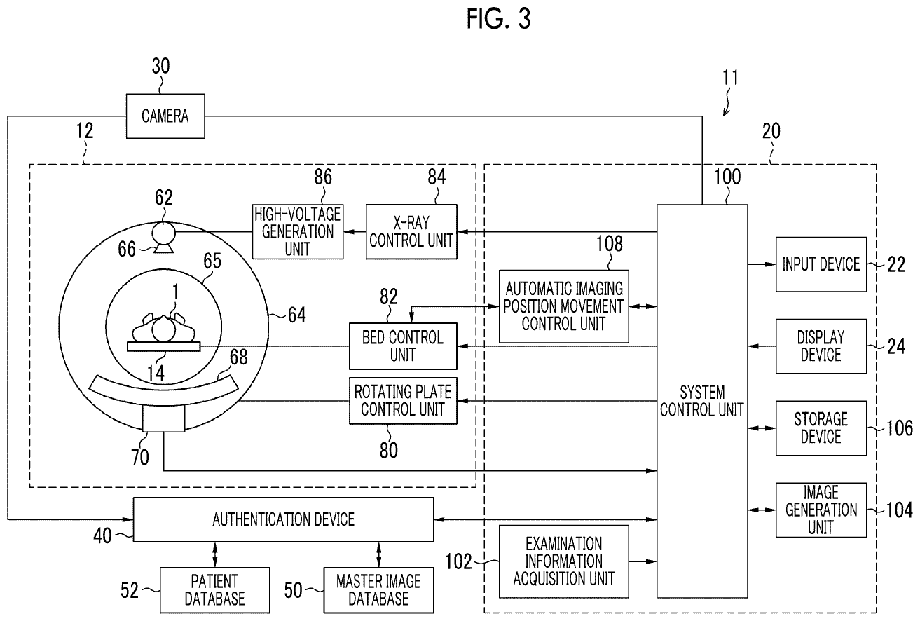

The scan gantry unit 12 emits X-rays to a subject placed on a top plate of the bed 14, detects the X-rays transmitted through the subject, and captures a medical image used for diagnosis or the like of the subject. It should be noted that the subject is designated by reference numeral 1 and is shown in FIG. 3. In addition, the top plate of the bed 14 is designated by reference numeral 14A and is shown in FIG. 2.

The examination control device 20 controls the operation of the scan gantry unit 12 and the bed 14. The examination control device 20 generates a tomographic image using digital data representing a spatial distribution of the X-rays transmitted from the scan gantry unit 12. The examination control device 20 performs various communication processes, data storage processes, and displays of processing results such as the tomographic image.

The camera 30 images the subject placed on the bed 14 to generate a camera image of the subject. The camera 30 comprises an imaging optical system, an image sensor, and a signal processing circuit. Here, the term “image” may include image data, which is an electric signal of the image.

The imaging optical system may be configured by combining a shutter, a stop, and a plurality of lenses. A CCD image sensor, a CMOS image sensor, or the like is applied to the image sensor. Note that CCD is an abbreviation for Charge Coupled Device, and CMOS is an abbreviation for Complementary Metal Oxide Semiconductor. The signal processing circuit generates a camera image of the subject based on the electric signal output from the image sensor.

The camera image of the subject is transmitted to the examination control device 20. The examination control device 20 analyzes the camera image of the subject and performs movement control of the bed 14 based on an analysis result.

The camera image of the subject is transmitted to the authentication device 40. The authentication device 40 performs personal authentication of the subject based on the camera image of the subject. Details of the personal authentication of the subject will be described below.

The authentication device 40 acquires a master image of the subject used for the personal authentication of the subject from the master image database 50. The master image database 50 stores the master image for each subject in association with subject identification information. A face image for each subject may be applied as the master image for each subject. The master image database 50 may store a plurality of master images having different imaging dates and times.

For example, in a case in which the subject is a first visit, a face image of a My Number card presented at a reception of a medical institution may be scanned, and a face image of the subject generated from scan data of the face image may be registered in the master image database 50 as the master image of the subject. A face image of the subject acquired by imaging the subject using an imaging apparatus provided at a reception of a medical institution or the like may be registered in the master image database 50 as the master image of the subject.

The authentication device 40 acquires patient information from the patient database 52. The patient database 52 stores patient information such as a visit history and an examination history for each patient in association with identification information such as a name of the patient. It should be noted that the patient information of the patient who is an examination target may be referred to as subject information.

The authentication device 40, the master image database 50, and the patient database 52 may be provided in a server device of a medical institution and may be electrically connected to a management system of the medical institution in a communicable manner.

It should be noted that the X-ray CT apparatus 11 is an example of a medical image capturing apparatus that generates medical image capturing data of a subject according to the present disclosure and is an example of a computed tomography imaging apparatus. The authentication device 40 is an example of an imaging support device according to the present disclosure. The personal authentication is an example of the personal identification according to the present disclosure.

### Configuration Example of X-ray CT Apparatus

FIG. 2 is a perspective view schematically showing a configuration of the X-ray CT apparatus shown in FIG. 1. The X-ray CT apparatus 11 comprises the scan gantry unit 12, the bed 14, and an operation unit 16. The scan gantry unit 12 and the bed 14 are disposed in an imaging room 17A covered with a shielding material that blocks X-rays, and the operation unit 16 is disposed in an operation room 17B. X shown in FIG. 2 represents a width direction of the bed 14 orthogonal to a traveling direction of the bed 14, and Y represents the traveling direction of the bed 14. Z represents a lifting direction of the bed 14.

The width direction of the bed 14 and the traveling direction of the bed 14 may be parallel to a disposition surface on which the X-ray CT apparatus 11 is disposed, and the lifting direction of the bed 14 may be a direction orthogonal to a surface on which the X-ray CT apparatus is disposed.

The term “orthogonal” in the present specification is not limited to intersecting at an angle of exactly 90 degrees, and may include intersecting at an angle of less than 90 degrees or more than 90 degrees, which can be treated in the same manner as strict orthogonality. In addition, the term “parallel” is not limited to strict parallelism, and may include substantial parallelism that can be treated in the same manner as strict parallelism.

The bed 14 is configured to be movable automatically or manually. FIG. 2 shows a state in which the bed 14 is set at a position where a medical image is imaged for a subject. The bed 14 comprises a top plate 14A on which the subject is placed in a lying-down state. The top plate 14A is supported to be movable in each of the traveling direction, the lifting direction, and the width direction by using a bed driving device.

The subject placed on the top plate 14A moves from a position outside the scan gantry unit 12 to an imaging position in the imaging space 12A in response to the movement of the top plate 14A from a position outside the scan gantry unit 12 to the imaging position in the imaging space 12A.

The operation unit 16 comprises the examination control device 20, the input device 22, and the display device 24. The examination control device 20 transmits a control signal to each of various control units of the scan gantry unit 12 to control each of the various control units.

The camera 30 is disposed on a ceiling of the imaging room 17A. The camera 30 images the subject placed on the top plate 14A of the bed 14 to generate the camera image of the subject. The position and the posture of the camera 30 are fixed to the ceiling. The position and the posture of the camera 30 are adjusted in advance such that the entire subject placed on the top plate 14A is within an angle of view.

It should be noted that the authentication device 40, the master image database 50, and the patient database 52 shown in FIG. 1 are not shown in FIG. 2. The authentication device 40, the master image database 50, and the patient database 52 may be disposed in the operation room 17B or may be disposed outside the operation room 17B.

### Electric Configuration of X-ray CT Apparatus

FIG. 3 is a functional block diagram showing an electric configuration of the X-ray CT apparatus shown in FIG. 2. The scan gantry unit 12 comprises an X-ray source 62, a rotating plate 64, a collimator 66, an X-ray detector 68, a data collection unit 70, and the bed 14. The scan gantry unit 12 comprises a rotating plate control unit 80, a bed control unit 82, an X-ray control unit 84, and a high-voltage generation unit 86.

The X-ray source 62 emits X-rays to a subject 1 placed on the bed 14. Examples of the X-ray source 62 include an X-ray tube device. The collimator 66 limits an irradiation range of the X-rays. The rotating plate 64 has an opening portion 65 into which the subject 1 placed on the bed 14 enters, and is mounted with the X-ray source 62 and the X-ray detector 68, and rotates the X-ray source 62 and the X-ray detector 68 around the subject 1.

The X-ray detector 68 is disposed at a position facing the X-ray source 62. The X-ray detector 68 comprises a plurality of detection elements that detect the X-rays transmitted through the subject 1, and is a device that detects a spatial distribution of the X-rays, and functions as a detection unit that detects a signal obtained from the subject 1. Detection elements of the X-ray detector 68 are arranged two-dimensionally in a rotation direction and a rotation axis direction of the rotating plate 64. The data collection unit 70 collects the spatial distribution of the X-rays detected by the X-ray detector 68 as digital data.

The rotating plate control unit 80 controls the rotation and the inclination of the rotating plate 64. The bed control unit 82 controls the movement of the bed 14 and the top plate 14A. The movement of the bed 14 and the top plate 14A includes forward movement, backward movement, movement in the width direction, and lifting. The high-voltage generation unit 86 generates a high voltage applied to the X-ray source 62. The X-ray control unit 84 controls an output of the high-voltage generation unit 86.

The examination control device 20 comprises a system control unit 100, an examination information acquisition unit 102, an image generation unit 104, a storage device 106, and an automatic imaging position movement control unit 108.

The examination information acquisition unit 102 acquires examination information. The examination information includes various types of information in the examination performed on the subject 1, such as identification information of the subject 1, an examination part, and an examination method.

The image generation unit 104 generates a tomographic image using the digital data collected via the data collection unit 70.

The storage device 106 stores the digital data collected using the data collection unit 70, the tomographic image generated using the image generation unit 104, a program executed by the system control unit 100, data used by the program, and the like. An HDD, an SSD, or the like may be applied to the storage device 106. It should be noted that HDD is an abbreviation for Hard Disk Drive. It should be noted that SSD is an abbreviation for Solid State Drive.

The system control unit 100 reads out a program corresponding to various functions of the medical image capturing system 10 from the storage device 106 and executes various programs to realize various functions. That is, the system control unit 100 controls various processing units such as the rotating plate control unit 80, the bed control unit 82, and the X-ray control unit 84.

The system control unit 100 acquires various types of input information transmitted from the input device 22 and transmits a control signal corresponding to the input information to each unit. The system control unit 100 transmits a display signal representing information to be displayed on the display device 24 to the display device 24.

The system control unit 100 acquires the camera image generated by imaging the subject 1 placed on the bed 14 using the camera 30 from the camera 30. The system control unit 100 transmits the camera image to the automatic imaging position movement control unit 108.

The automatic imaging position movement control unit 108 acquires the camera image transmitted from the system control unit 100 and detects a position of an imaging part of the subject 1 in the camera image. A coordinate value in the camera image may be applied to the position of the imaging part of the subject 1. A coordinate value in a real space in which the coordinate value in the camera image is converted may be applied to the position of the imaging part of the subject 1.

The automatic imaging position movement control unit 108 performs automatic imaging position movement control of the bed 14 to move the bed 14 to a position at which the imaging part of the subject 1 matches an imaging center of the imaging space 12A. That is, the automatic imaging position movement control unit 108 transmits an automatic imaging position movement control signal to the bed control unit 82. The bed control unit 82 moves the bed 14 and the top plate 14A based on the automatic imaging position movement control signal. It should be noted that the term “match” may include substantial match having a deviation within a predetermined range.

The high-voltage generation unit 86 generates a tube voltage to be applied to the X-ray source 62 based on an imaging condition of the medical image set via the input device 22. The X-ray source 62 to which the tube voltage is applied emits X-rays corresponding to the imaging condition to the subject 1.

The X-ray detector 68 detects the transmitted X-rays that are the X-rays emitted from the X-ray source 62 and transmitted through the subject 1, using the plurality of detection elements, and acquires a spatial distribution of the transmitted X-rays.

The rotating plate 64 is controlled to perform a rotation operation using the rotating plate control unit 80. That is, the rotating plate 64 rotates based on the imaging condition acquired via the input device 22, particularly, the setting of the rotation speed.

The bed 14 is controlled using the bed control unit 82. That is, the bed 14 moves relative to the rotating plate 64 to a region of an imaging field of view through which the transmitted X-rays are detected for the imaging part of the subject 1 that is set.

During the rotation of the rotating plate 64, the emission of the X-rays using the X-ray source 62 and the detection of the X-rays using the X-ray detector 68 are repeated, and projection data that is an X-ray projection image of the subject 1 is measured at various projection angles. The projection data is associated with a view representing each projection angle and a detection element number of the X-ray detector 68. The detection element number may include a channel number and a column number.

The measured projection data is transmitted to the image generation unit 104. The image generation unit 104 performs back projection processing on the plurality of pieces of projection data to generate the tomographic image. The generated tomographic image may be displayed on the display device 24 as the medical image or may be stored in the storage device 106.

The camera 30 images the subject 1 placed on the bed 14 from above the subject 1. A position at which the camera 30 is fixedly disposed is adjusted such that the entire body of the subject 1 is within the angle of view of the camera 30.

The camera image of the subject 1 captured and acquired using the camera 30 may be displayed on the display device 24. The operator of the operation room can visually recognize the camera image of the subject 1 and can check the state of the subject 1. The camera image of the subject 1 may be stored in the storage device 106.

The camera image of the subject 1 is used for movement control of the bed 14 to the imaging space 12A of the medical image. In addition, the camera image of the subject 1 is transmitted to the authentication device 40 and is used for checking the subject 1.

### Configuration Example of Authentication Device According to First Embodiment

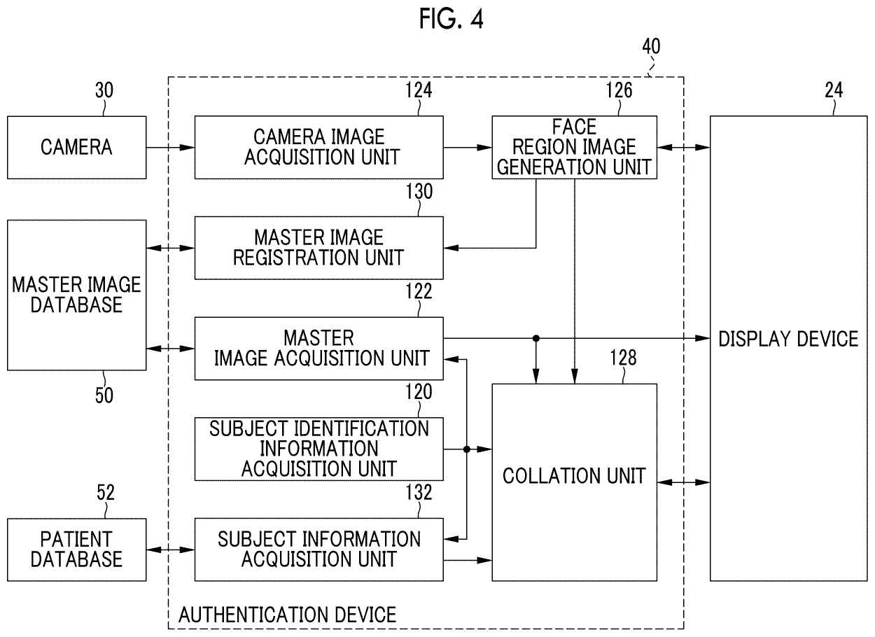

FIG. 4 is a functional block diagram showing an electric configuration of the authentication device according to the first embodiment. The authentication device 40 performs the personal authentication of the subject 1 that determines whether or not the subject 1 placed on the bed 14 shown in FIG. 2 corresponds to the examination target that is understood from the subject identification information.

Specifically, the face information of the subject 1 and the master image stored in advance are collated, and it is determined whether or not the face information of the subject 1 matches the master image of the subject 1 associated with the subject identification information of the subject 1.

A face region image of the subject 1 extracted from the camera image of the subject 1 acquired using the camera 30 fixedly disposed on the ceiling of the imaging room 17A may be applied as the face information of the subject 1.

For example, in a case in which the similarity between the face region image of the subject 1 and the master image of the subject 1 exceeds a predetermined value, it is determined that the face information of the subject 1 matches the master image of the subject 1. It should be noted that the face information of the subject 1 is an example of the first face information according to the present disclosure. The face region image of the subject 1 is an example of the first face region image according to the present disclosure.

The authentication device 40 comprises a subject identification information acquisition unit 120. The subject identification information acquisition unit 120 acquires the subject identification information of the subject 1. The subject identification information includes information used for identifying the subject 1, such as a name of the subject 1 and a patient number assigned to the subject 1. The subject identification information acquisition unit 120 may acquire the subject identification information of the subject 1 described in the examination order or the like from the examination control device 20 shown in FIG. 3.

The authentication device 40 comprises a master image acquisition unit 122. The master image acquisition unit 122 acquires the master image of the subject 1 registered in advance from the master image database 50 based on the subject identification information of the subject 1.

The master image of the subject 1 includes the face of the subject 1. The master image of the subject 1 may include a part around the face, such as a head and an upper body of the subject 1. In a case in which a plurality of master images are stored for one subject 1, the authentication device 40 may acquire one master image selected from the plurality of master images. Examples of the plurality of master images include a plurality of master images having different imaging times. The authentication device 40 may display the master image on the display device 24.

In a case in which the master image associated with the subject identification information matching the subject identification information of the subject 1 does not exist in the master image database 50, the master image acquisition unit 122 may display information indicating that the master image does not exist on the display device 24.

The authentication device 40 comprises a camera image acquisition unit 124. The camera image acquisition unit 124 acquires the camera image of the subject 1 captured using the camera 30. The camera image acquisition unit 124 may acquire a still image as the camera image or may acquire a moving image. The acquisition of the moving image may be the acquisition of one or more frame images constituting the moving image.

The camera image acquisition unit 124 may acquire a plurality of still images or a plurality of frame images. The camera image acquisition unit 124 may acquire one still image or one frame image selected from the plurality of still images or the plurality of frame images.

The authentication device 40 comprises a face region image generation unit 126. The face region image generation unit 126 extracts a face region including the face of the subject 1 from the camera image including the entire body of the subject 1 lying on the bed 14, and generates the face region image of the subject 1.

For example, the face region image generation unit 126 may extract the face region of the subject 1 from the camera image, cut out the extracted face region of the subject 1 from the camera image, and generate the face region image of the subject 1.

The face region of the subject 1 includes the face of the subject 1. The face region of the subject 1 may include a part of an upper body of the subject 1, such as a head of the subject 1 and a shoulder of the subject 1. The authentication device 40 may display the face region image of the subject 1 on the display device 24.

The authentication device 40 comprises a collation unit 128. The collation unit 128 acquires the master image acquired based on the subject identification information of the subject 1 and the face region image of the subject 1 based on the camera image of the subject 1, and performs collation between the two images.

The collation unit 128 may perform image processing on the face region image to be collated, such as processing of matching a size between the master image to be collated and the face region image, processing of matching a resolution, processing of matching a brightness, and processing of matching a tint.

The collation unit 128 determines whether or not the master image matches the face region image. A similarity between the master image and the face region image may be applied to the determination of the match or the mismatch between the master image and the face region image. The collation unit 128 may display the determination result on the display device 24.

The authentication device 40 comprises a master image registration unit 130. The master image registration unit 130 registers the face region image generated from the camera image in the master image database 50 as the master image. The master image registration unit 130 may add accessory information such as the subject identification information and imaging information such as an imaging date to the face region image.

The master image registration unit 130 may register the face region image in the master image database 50 as the master image in a case in which the master image of the subject 1 does not exist, in a case in which a period from an imaging date of the master image of the subject 1 has elapsed, and the like.

The authentication device 40 comprises a subject information acquisition unit 132. The subject information acquisition unit 132 acquires subject information related to the subject 1 associated with the subject identification information from the patient database 52.

FIG. 4 shows an aspect in which the master image database 50 is provided separately from the patient database 52, but the master image database 50 may be a component of the patient database 52.

It should be noted that the camera image of the subject 1 is an example of the captured image of the subject according to the present disclosure, and the master image of the subject 1 is an example of the master face information of the subject according to the present disclosure. The master image database 50 is an example of a master face information registration device according to the present disclosure.

### Hardware Configuration Example of Authentication Device

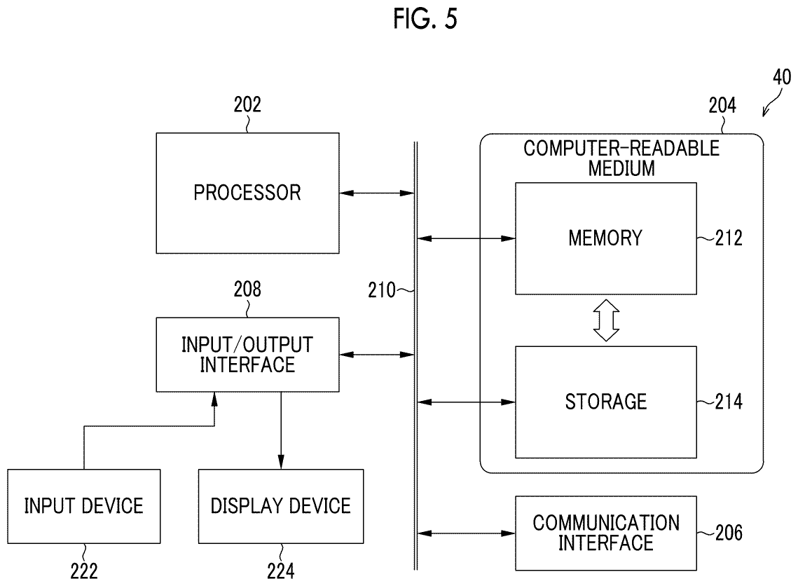

FIG. 5 is a block diagram showing a hardware configuration example of the electric configuration of the authentication device according to the first embodiment. One or more computers are applied to the authentication device 40. The computer that functions as the authentication device 40 executes a program to realize various functions of the authentication device 40.

The computer may be a general-purpose computer such as a personal computer or a computer for a specific use such as a server computer. Any computer may be a system such as a workstation or may be other hardware elements capable of executing a program, such as a virtual machine.

At least a part of the functions of the authentication device 40 may be realized by using cloud computing. At least a part of the functions of the authentication device 40 may be provided as SaaS. It should be noted that SaaS is an abbreviation for Software as a Service.

The authentication device 40 comprises a processor 202, a computer-readable medium 204, a communication interface 206, an input/output interface 208, and a bus 210.

The processor 202 is connected to the computer-readable medium 204, the communication interface 206, the input/output interface 208, an input device 222, and a display device 224 via the bus 210. The input device 222 shown in FIG. 5 may be used in common with the input device 22 shown in FIG. 1, and the display device 224 shown in FIG. 5 may be used in common with the display device 24 shown in FIG. 1.

The computer-readable medium 204 comprises a memory 212 as a main storage device and a storage 214 as an auxiliary storage device. The memory 212 includes a RAM. The memory 212 may include a ROM.

The storage 214 may be, for example, a hard disk drive, a solid state drive, or a combination of a plurality of these. In addition, the storage 214 may include an external storage device such as a removable medium.

It should be noted that RAM is an abbreviation for Random Access Memory, and ROM is an abbreviation for Read Only Memory.

The memory 212 stores a program, data, and the like for realizing various functions of the authentication device 40. The processor 202 executes the programs stored in the memory 212 to realize various functions. The processor 202 integrally controls each unit of the authentication device 40 and performs various types of processing.

The communication interface 206 includes a communication interface or the like that can be connected to an electric communication line such as a local area network. The communication interface 206 may be a wireless format or a wired format.

The input/output interface 208 includes a connection interface or the like that can be connected to an external device. As the connection interface that is connectable to an external device, for example, a universal serial bus or HDMI (HDMI is a registered trademark) can be applied. It should be noted that HDMI is an abbreviation for High-Definition Multimedia Interface.

Examples of the input device 222 include a pointing device such as a keyboard and a mouse. The input device 222 may include a numeric keypad, various switch buttons, and the like. The input device 222 may include a voice input device. The input device 222 may be a touch panel type input device that is configured integrally with a display screen of the display device 224.

Various types of information are displayed on the display device 224 in addition to the camera image acquired using the camera 30. The display device 224 is used as a part of the user interface in a case where an input from the input device 222 is accepted. The present disclosure is not limited to one display device 224, and a form of a multi-display comprising a plurality of display devices 224 can be used. It should be noted that the organic EL may be referred to as OEL using an abbreviation for Organic Electro-Luminescence.

The hardware configuration of the electric configuration of the authentication device 40 shown in FIG. 5 is applied as the hardware configuration of the electric configuration of the examination control device 20 shown in FIG. 3 and the like, an authentication device according to a second embodiment, and an authentication device according to a third embodiment. It should be noted that the processor 202 of the authentication device 40 is an example of the first processor according to the present disclosure, and the computer-readable medium 204 of the authentication device 40 is an example of the first memory according to the present disclosure. In addition, the processor 202 of the examination control device 20 is an example of the second processor according to the present disclosure, and the computer-readable medium 204 of the authentication device 40 is an example of the second memory according to the present disclosure.

### Procedure of Authentication Method According to First Embodiment

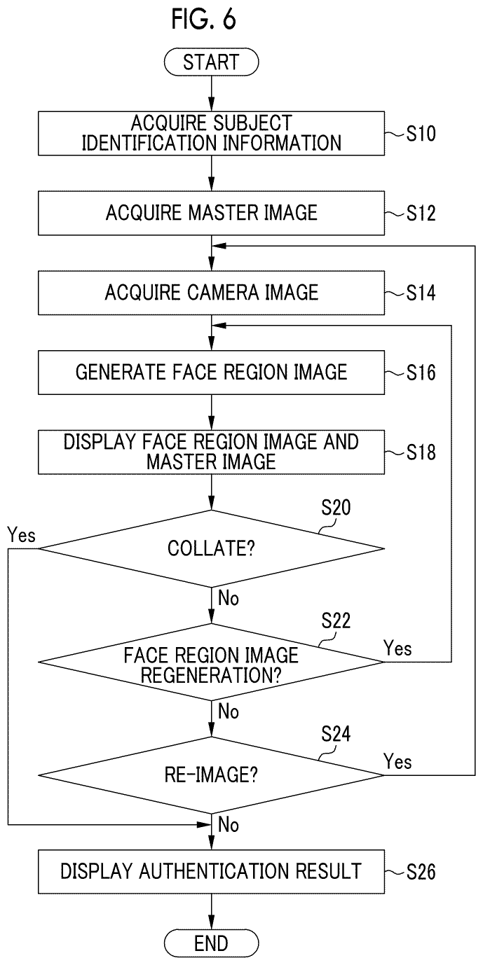

FIG. 6 is a flowchart showing a procedure of the authentication method according to the first embodiment. The authentication method in which the procedure is shown in FIG. 6 is realized by the computer that functions as the authentication device 40 or the like executing the program stored in the memory 212 shown in FIG. 5.

In step S10, the subject identification information acquisition unit 120 shown in FIG. 4 acquires the subject identification information of the subject 1. In step S12, the master image acquisition unit 122 acquires the master image of the subject 1 associated with the subject identification information of the subject 1 from the master image database 50.

In step S14, the camera image acquisition unit 124 acquires the camera image of the subject 1. The steps S12 and S14 may be executed in an order that is different from the order or may be executed in parallel.

In step S16, the face region image generation unit 126 generates the face region image of the subject 1. In step S18, the master image acquisition unit 122 displays the master image acquired in step S12 on the display device 24. In addition, in step S18, the face region image generation unit 126 displays the face region image of the subject 1 on the display device 24.

In step S20, the collation unit 128 collates the master image of the subject 1 with the face region image of the subject 1. In step S20, in a case in which the face region image of the subject 1 matches the master image of the subject 1, a Yes determination is made, and the process proceeds to step S26.

On the other hand, in step S20, in a case in which the face region image of the subject 1 does not match the master image of the subject 1, a No determination is made, and the process proceeds to step S22. In step S22, the collation unit 128 determines whether or not to regenerate the face region image of the subject 1. For example, the collation unit 128 may determine to regenerate the face region image of the subject 1 in a case in which the operator who has checked the face region image of the subject 1 has input the information representing the regeneration of the face region image of the subject 1.

In step S22, in a case in which it is determined to execute the regeneration of the face region image of the subject 1, a Yes determination is made, and the process proceeds to step S16. In step S16, the face region image generation unit 126 regenerates the face region image of the subject 1 from the camera image of the subject 1. In the regeneration of the face region image, a processing condition for the camera image of the subject 1 may be changed.

In a case in which the Yes determination is made in step S22, each step from step S14 to step S24 is repeatedly executed as appropriate until the Yes determination is made in step S20. The number of repetitions of each step may be appropriately set.

On the other hand, in step S22, in a case in which it is determined not to execute the regeneration of the face region image of the subject 1, a No determination is made, and the process proceeds to step S24. In step S24, the collation unit 128 determines whether or not to re-image the subject 1.

In step S24, as an example in a case in which it is determined that the re-imaging is necessary, a case is exemplified in which it is difficult to generate the face region image in which appropriate collation with the master image is realized in a case in which the processing condition is changed in a case of generating the face region image.

In step S24, in a case in which it is determined not to execute the re-imaging of the subject 1, a No determination is made, and the process proceeds to step S26. On the other hand, in step S24, in a case in which it is determined to execute the re-imaging of the subject 1, a Yes determination is made.

In a case of Yes in step S24, the collation unit 128 displays on the display device 24 that the re-imaging of the subject 1 is performed. In addition, in a case of Yes in step S24, the process proceeds to step S14, and each step from step S14 to step S26 is repeatedly executed as appropriate until the Yes determination is made in step S20 or the No determination is made in step S24. The number of repetitions of each step may be appropriately set.

In a case in which the re-imaging of the subject 1 is executed, imaging parameters of the subject 1 may be changed, and a posture of the subject 1, such as an orientation of the face of the subject 1 lying on the bed 14, and a position of the subject 1 on the bed 14 may be changed.

In step S26, the collation unit 128 displays a result of the personal authentication based on the collation result on the display device 24. In a case in which the Yes determination is made in step S20, the face region image of the subject 1 matches the master image of the subject 1, and a result of the personal authentication representing the success is displayed on the display device 24.

In a case in which the No determination is made in step S24, a result of the personal authentication representing the failure is displayed on the display device 24. In a case in which the result of the personal authentication representing the failure is displayed, a cause of the failure may be displayed.

In a case in which the personal authentication of the subject 1 is successful, the process proceeds to the capturing of the medical image for the subject 1. On the other hand, in a case in which the personal authentication of the subject 1 is unsuccessful, the examination order is checked or the like is performed. It should be noted that the personal authentication method in which the procedure is shown in FIG. 6 is an example of the personal identification method according to the present disclosure.

### Example of Master Image Display Screen

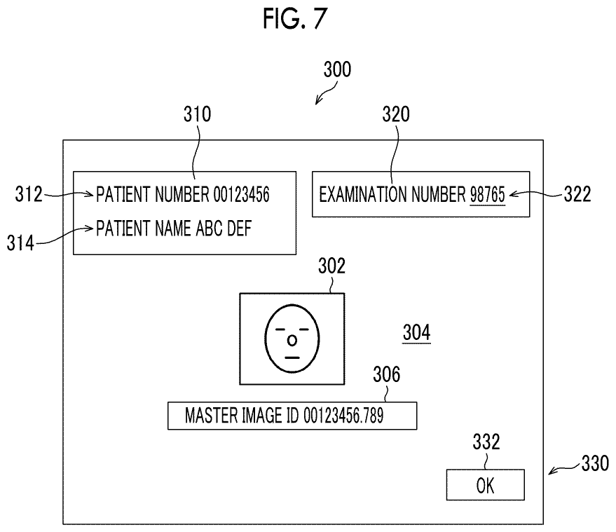

FIG. 7 is a schematic diagram of a master image display screen. A master image display screen 300 shown in FIG. 7 is displayed on the display device 24 shown in FIG. 1 and the like. The master image display screen 300 includes a master image display region 304 in which a master image 302 is displayed.

The master image display region 304 shown in FIG. 7 includes master image ID information 306 representing a master image ID corresponding to the master image 302. In the master image display region 304, an imaging condition of the master image 302, such as an imaging date of the master image and imaging parameters applied to the imaging of the master image, may be displayed. It should be noted that ID is an abbreviation for identification.

The master image display screen 300 includes a subject information display region 310. In the subject information display region 310, a patient number 312 that is an identification number of the subject 1 and a patient name 314 that is a name of the subject 1 are displayed. The patient number 312 and the patient name 314 may be information read out from an electronic medical record.

The master image display screen 300 includes an examination number display region 320. In the examination number display region 320, an examination number 322 read out from the examination order is displayed.

The master image display screen 300 includes a button display region 330. The button display region 330 includes an OK button 332. The OK button 332 is operated in a case in which the display of the master image display screen 300 ends.

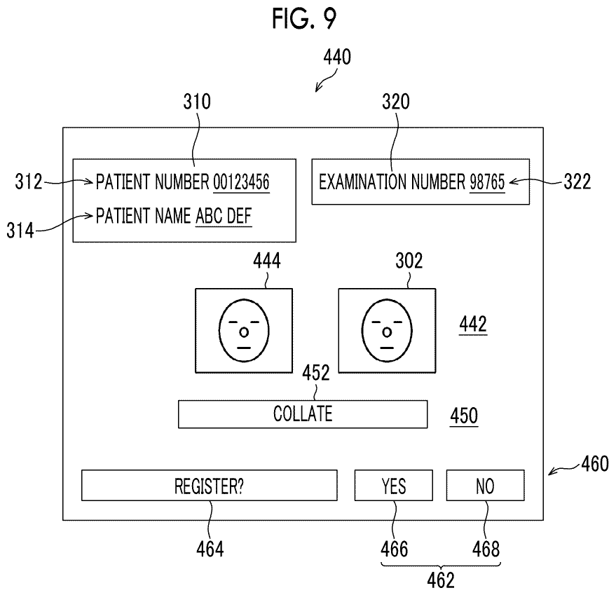

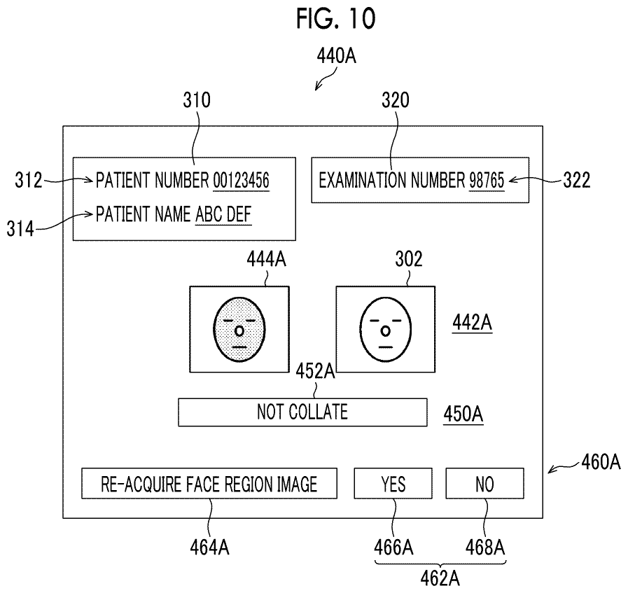

It should be noted that the master image display screen 300 may display a menu bar, a task bar, a toolbar, and the like. The same applies to other display screens such as the collation result display screen shown in FIGS. 9 and 10.

FIG. 7 shows an example of an arbitrary number sequence as the number such as the patient number 312, but the patient number 312 may include a character other than the number, such as an alphabet, and the number of digits may be appropriately determined. The same applies to the examination number 322 and the master image ID information 306.

In addition, FIG. 7 shows a character string including a plurality of arbitrary characters as the patient name 314. The patient name 314 may be applied with Kanji, Kana, or an alphabet, and may be an appropriate combination of Kanji or the like.

### Example of Camera Image of Subject

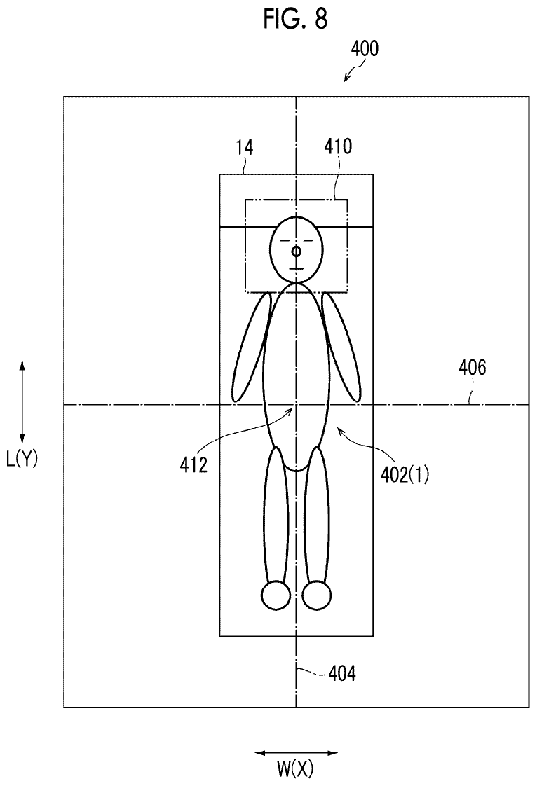

FIG. 8 is a schematic diagram of a camera image. FIG. 8 schematically shows a camera image 400 of the subject 1 that is imaged and acquired in a case in which the subject 1 placed in a state of lying on the bed 14 is imaged using the camera 30 fixedly disposed on the ceiling of the imaging room 17A shown in FIG. 2.

In a case in which the subject 1 is imaged using the camera 30, a through image including the subject 1 may be displayed on the display device 24. A horizontal center line 404 representing a center in a horizontal direction of the camera image 400 and a vertical center line 406 representing a center position in a vertical direction of the camera image may be displayed on the through image. An intersection of the horizontal center line 404 and the vertical center line 406 represents a center position 412 of the camera image 400.

An arrow line to which reference numeral W is assigned represents a horizontal direction of the camera image 400. An arrow line to which reference numeral L is assigned represents a vertical direction of the camera image 400. The horizontal direction of the camera image 400 corresponds to a width direction of the bed 14 in a real space represented by reference numeral X. The vertical direction of the camera image 400 corresponds to a traveling direction of the bed 14 in a real space represented by reference numeral Y.

The camera 30 is adjusted to a posture in which the optical axis passes through the center position 412 of the bed 14. The adjustment of the posture of the camera 30 includes the adjustment of the position of the camera 30, the adjustment of the direction in which the optical axis of the camera 30 faces, and the adjustment of the angle of view of the camera 30.

The camera image 400 includes a subject image 402 representing the entire body of the subject 1 placed on the bed 14. The camera image 400 includes a background image of the subject 1 such as the bed 14.

The face region image generation unit 126 shown in FIG. 4 extracts a face region 410 of the subject image 402 from the camera image 400 shown in FIG. 8, cuts out the extracted face region 410 from the camera image 400, and generates the face region image of the subject 1.

As the extraction of the face region, feature region extraction processing of extracting the face region 410 of the subject image 402 from the camera image 400 as a feature region may be applied. In the extraction of the face region 410, a position of the face region 410 in the camera image 400 may be detected according to whether the body position of the subject 1 is head first or foot first. The body position of the subject 1 may be read out from the examination order.

In the extraction of the face region 410, a part of the subject 1, such as a head, a shoulder, and an arm, may be detected, and a position of the face region 410 in the camera image 400 may be detected based on the position of the head or the like. In the extraction of the face region 410, the position of the face region 410 in the camera image 400 may be understood with reference to the height or the like of the subject 1 based on the center position 412 of the camera image 400.

### Display Example of Collation Result

FIG. 9 is a schematic diagram of a collation result display screen in a case in which the face region image matches the master image. A collation result display screen 440 shown in FIG. 9 is displayed on the display device 24 in a case in which a face region image 444 of the subject 1 matches the master image 302 of the subject 1 in step S20 of FIG. 6. The match between the face region image 444 of the subject 1 and the master image 302 of the subject 1 may mean that the person in the face region image 444 and the person in the master image 302 are the same person.

The collation result display screen 440 includes the subject information display region 310 and the examination number display region 320 as in the master image display screen 300 shown in FIG. 7. The collation result display screen 440 includes an image display region 442. The image display region 442 includes the master image 302 and the face region image 444.

The collation result display screen 440 includes a collation result display region 450. The collation result display region 450 includes a collation result 452. FIG. 9 shows character information representing that the face region image 444 of the subject 1 matches the master image 302 of the subject 1 as the collation result 452. A pop-up may be applied to the collation result 452.

The collation result display screen 440 includes a button display region 460. The button display region 460 includes one or more buttons 462 operated by the operator and processing information 464 representing processing corresponding to the operation of the button 462.

FIG. 9 shows character information representing processing of registering the master image 302 as the processing information 464. In addition, FIG. 9 shows a button 466 operated in a case in which the processing is performed and a button 468 operated in a case in which the processing is not performed.

In a case in which the operator visually recognizes the collation result display screen 440, the face region image 444 of the subject 1 may be registered as a new master image in a case in which the face region image 444 of the subject 1 matches the master image 302 of the subject 1.

In a case in which the new master image is registered, the new master image may be added and registered without erasing the master image 302 already stored, or the master image 302 already stored may be updated to the new master image.

FIG. 10 is a schematic diagram of a collation result display screen in a case in which the face region image does not match the master image. A collation result display screen 440A shown in FIG. 10 is displayed on the display device 24 in a case in which a face region image 444A of the subject 1 does not match the master image 302 of the subject 1 in step S20 of FIG. 6.

The collation result display screen 440A includes the subject information display region 310 and the examination number display region 320 as in the master image display screen 300 shown in FIG. 7. The collation result display screen 440A includes an image display region 442A. The image display region 442A includes the master image 302 and the face region image 444A.

The collation result display screen 440A includes a collation result display region 450A. The collation result display region 450A includes a collation result 452A. FIG. 10 shows character information representing that the face region image 444A of the subject 1 does not match the master image 302 of the subject 1 as the collation result 452A. A pop-up may be applied to the collation result 452A.

The collation result display screen 440A includes a button display region 460A. The button display region 460A includes one or more buttons 462A operated by the operator and processing information 464A representing processing corresponding to the operation of the button 462A.

FIG. 10 shows character information representing processing of re-acquiring the face region image as the processing information 464A. In addition, FIG. 10 shows a button 466A operated in a case in which the processing is performed and a button 468A operated in a case in which the processing is not performed.

In a case in which the button 466A corresponding to the execution of the processing is operated, the process proceeds to step S16, and the face region image of the subject 1 is generated from the camera image 400 of the subject 1 that has already been acquired. On the other hand, in a case in which the button 468A corresponding to the non-execution of the processing is operated, the process proceeds to step S24, and it is determined whether or not to re-acquire the camera image 400.

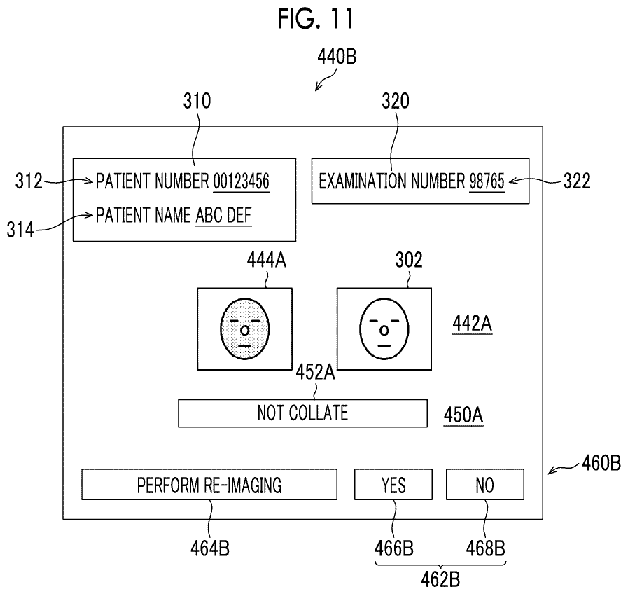

FIG. 11 is a schematic diagram of a collation result display screen in a case in which the face region image does not match the master image. A collation result display screen 440B shown in FIG. 11 is displayed in step S24 of FIG. 6.

The collation result display screen 440B includes the subject information display region 310 and the examination number display region 320 as in the master image display screen 300 shown in FIG. 7. The collation result display screen 440B includes an image display region 442A as in the collation result display screen 440A shown in FIG. 10. The image display region 442A includes the master image 302 and the face region image 444A.

The collation result display screen 440B includes a button display region 460B. The button display region 460B includes one or more buttons 462B operated by the operator and processing information 464B representing processing corresponding to the operation of the button 462B, in which character information representing processing of performing the re-imaging is applied to the processing information 464B.

In a case in which the button 466B corresponding to the execution of the processing is operated, the process proceeds to step S14, and the subject 1 is re-imaged using the camera 30. On the other hand, in a case in which the button 468B corresponding to the non-execution of the processing is operated, the process proceeds to step S26, and the result of the personal authentication is displayed on the display device 24.

### Display Example of Personal Authentication Result

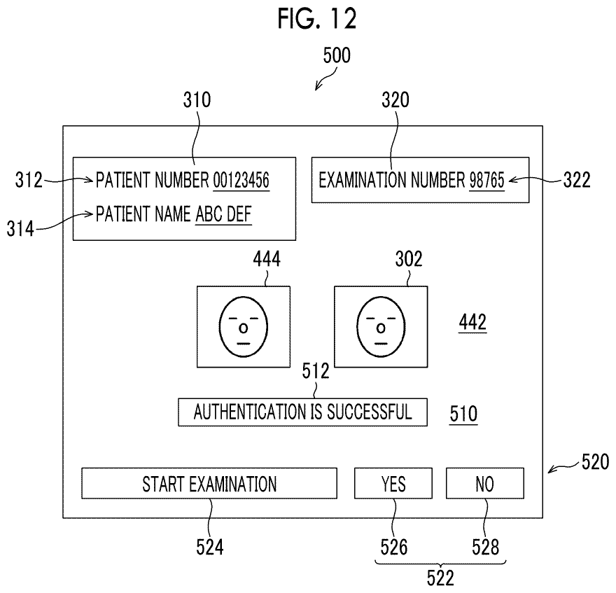

FIG. 12 is a schematic diagram of an authentication result display screen in a case in which the personal authentication is successful. An authentication result display screen 500 shown in FIG. 12 is displayed on the display device 24 in step S26 of FIG. 6.

The authentication result display screen 500 includes an authentication result display region 510 instead of the collation result display region 450 of the collation result display screen 440 shown in FIG. 9. The authentication result display region 510 includes an authentication result 512. FIG. 12 shows character information representing the success of the personal authentication as the authentication result 512.

In addition, the authentication result display screen 500 includes a button display region 520 instead of the button display region 460 shown in FIG. 9. The button display region 520 includes one or more buttons 522 operated by the operator and processing information 524 representing processing corresponding to the operation of the button 522.

FIG. 12 shows processing information 524 to which character information representing the start of the examination is applied, a button 526 operated in a case in which the processing is performed, and a button 528 operated in a case in which the processing is not performed.

The operator who visually recognizes the authentication result display screen 500 can understand that the personal authentication of the subject 1 is successful. The operator can operate the button 526 corresponding to the execution of the processing to start the capturing of the medical image for the subject 1. On the other hand, the operator can also operate the button 528 corresponding to the non-execution of the processing to prevent the capturing of the medical image for the subject 1 from being started.

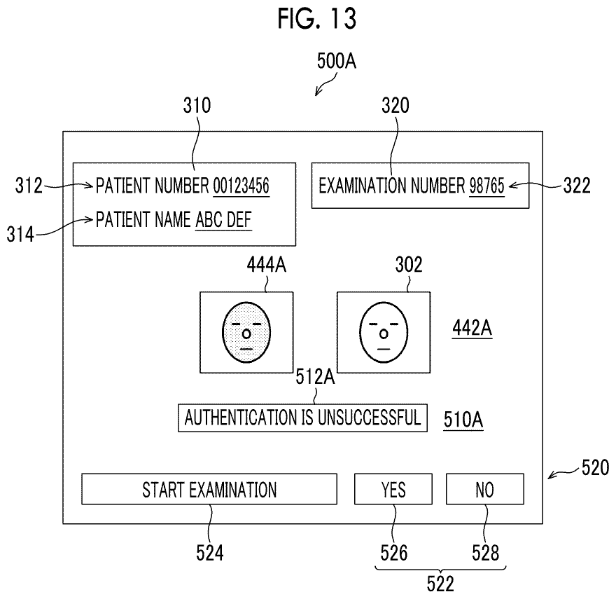

FIG. 13 is a schematic diagram of an authentication result display screen in a case in which the personal authentication is unsuccessful. An authentication result display screen 500A shown in FIG. 13 is displayed on the display device 24 in step S26 of FIG. 6.

The authentication result display screen 500A includes an authentication result display region 510A instead of the collation result display region 450A of the collation result display screen 440A shown in FIG. 10. The authentication result display region 510A includes an authentication result 512A. FIG. 13 shows character information representing the failure of the personal authentication as the authentication result 512A. In addition, the authentication result display screen 500A includes a button display region 520 as in the button display region 520 shown in FIG. 12.

The operator who visually recognizes the authentication result display screen 500A can understand that the authentication of the subject 1 is unsuccessful. The operator may operate the button 528 corresponding to the non-execution of the processing to continue the authentication of the subject 1 without starting the capturing of the medical image for the subject 1. On the other hand, the operator may operate the button 526 corresponding to the execution of the processing to start the capturing of the medical image for the subject 1.

### Actions and Effects of First Embodiment

The authentication device 40 and the authentication method according to the first embodiment can obtain the following effects.

[1] The subject 1 placed on the bed 14 is imaged using the camera 30 fixedly disposed on the ceiling of the imaging room 17A, and the camera image 400 of the subject 1 is acquired. The face region 410 of the subject 1 is extracted from the camera image 400 of the subject 1, and the face region image 444 of the subject 1 or the like is generated and acquired. It is determined whether or not the face region image 444 of the subject 1 or the like matches the master image 302 of the subject 1, and the result of the personal authentication of the subject 1 based on the determination result is displayed on the display device. As a result, suitable personal authentication for the subject 1 in the lying-down posture is realized.

[2] In a case in which the face region image 444A of the subject 1 does not match the master image 302 of the subject 1, the re-acquisition of the face region image 444 of the subject 1 or the like is performed. As a result, it is possible to determine whether or not the re-acquired face region image 444 of the subject 1 or the like matches the master image 302 of the subject 1.

[3] In a case in which the face region image 444A of the subject 1 does not match the master image 302 of the subject 1, the camera image 400 of the subject 1 is re-imaged. As a result, it is possible to determine whether or not the face region image 444 of the subject 1 or the like based on the re-imaged camera image 400 of the subject 1 matches the master image 302 of the subject 1.

[4] The result of the personal authentication of the subject 1 is displayed on the display device 24. As a result, the operator can understand the result of the personal authentication of the subject 1.

[5] The camera image 400 of the subject 1 used for deriving a control parameter in the automatic imaging position movement control of the bed 14 is used for the personal authentication of the subject 1 placed on the bed 14. As a result, the camera image 400 of the subject 1 applied to the automatic imaging position movement control of the bed 14 is used for the personal authentication of the subject 1 in the lying-down posture.

### Configuration Example of Authentication Device According to Second Embodiment

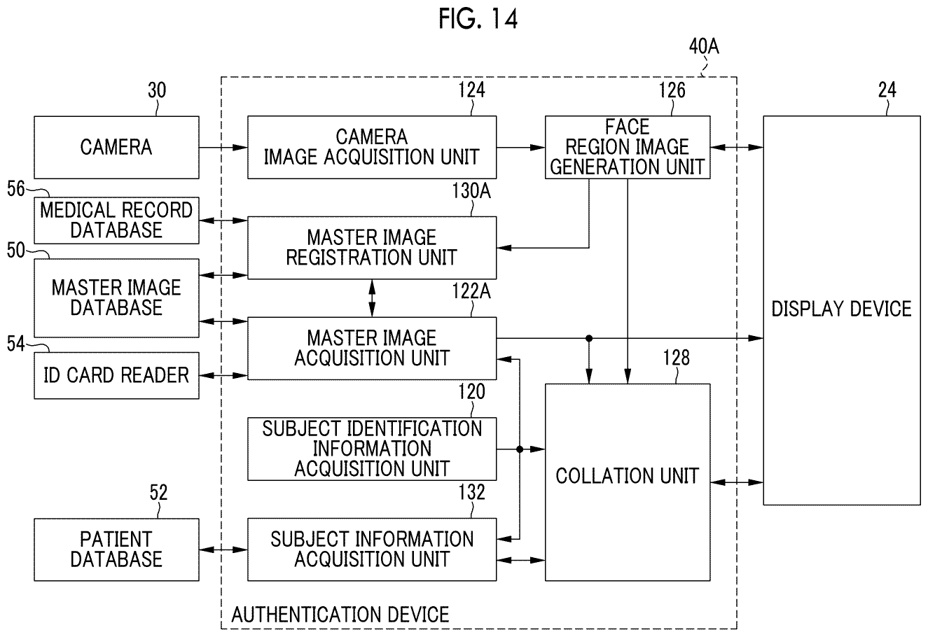

FIG. 14 is a functional block diagram showing an electric configuration of an authentication device according to a second embodiment. Hereinafter, the difference between the authentication device 40A according to the second embodiment and the authentication device 40 according to the first embodiment will be mainly described, and the description of the same components as those of the authentication device 40 according to the first embodiment will be appropriately omitted.

The authentication device 40A according to the second embodiment comprises a master image acquisition unit 122A instead of the master image acquisition unit 122 of the authentication device 40 shown in FIG. 4. In a case in which the master image of the subject 1 is not stored in the master image database 50, the master image acquisition unit 122A acquires scan data of a face image of the subject 1 included in the ID card using the ID card reader 54, generates a face region image of the subject 1 from the scan data of the face image of the subject 1, and acquires the face region image generated from the scan data of the face image of the subject 1 as the master image of the subject 1.

The scan data of the face image of the subject 1 may be data in which a region of the face image of the subject 1 is extracted from the scan data of the ID card of the subject 1, or may be data in which the face image of the subject 1 in the ID card of the subject 1 is read.

The authentication device 40A comprises a master image registration unit 130A instead of the master image registration unit 130 of the authentication device 40 shown in FIG. 4. The master image registration unit 130A registers the master image of the subject 1 based on the ID card of the subject 1, which is acquired using the master image acquisition unit 122A, in the master image database 50. The master image registration unit 130A associates the master image of the subject 1 based on the ID card of the subject 1 with the medical record information of the subject 1 stored in the medical record database 56.

Examples of the ID card of the subject 1 include a My Number card of the subject 1. The ID card of the subject 1 includes the face image of the subject 1, and the scan data of the face image of the subject 1 may be acquired.

The authentication device 40A may comprise a scan data processing unit that performs image processing such as resolution correction, color correction, and brightness correction on the scan data of the face image of the subject 1.

A hardware configuration of the electric configuration of the authentication device 40A according to the second embodiment is the same as the hardware configuration of the electric configuration of the authentication device 40 according to the first embodiment.

### Procedure of Authentication Method According to Second Embodiment

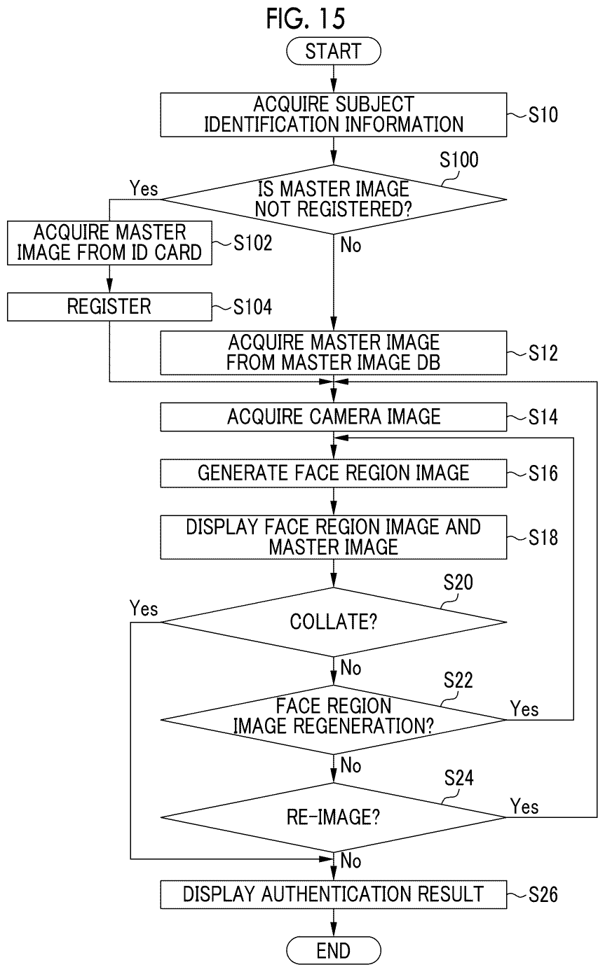

FIG. 15 is a flowchart showing a procedure of an authentication method according to the second embodiment. In the authentication method according to the second embodiment, steps S100, S102, and S104 are added to the authentication method according to the first embodiment in which the procedure is shown in FIG. 6.

In the authentication method according to the second embodiment, in a case in which the subject identification information is acquired in step S10, the process proceeds to step S100. In step S100, the master image acquisition unit 122A shown in FIG. 14 determines whether or not the master image of the subject 1 is registered in the master image database 50.

In step S100, in a case in which the master image of the subject 1 is registered in the master image database 50, a No determination is made, and the process proceeds to step S12. In step S12, the master image acquisition unit 122A acquires the master image of the subject 1 from the master image database 50.

On the other hand, in step S100, in a case in which the master image of the subject 1 is not registered in the master image database 50, a Yes determination is made, and the process proceeds to step S102. In step S102, the master image acquisition unit 122A acquires a face region image generated from the scan data of the face image included in the ID card of the subject 1 read using the ID card reader 54, as the master image of the subject 1.

In step S104, the master image registration unit 130A registers the face region image of the subject 1 based on the scan data of the face image of the subject 1, as the master image of the subject 1 in the master image database 50.

In a case in which the master image of the subject 1 is acquired in step S102 and the master image of the subject 1 is registered in the master image database 50 in step S104, the process proceeds to step S14. After each step from step S14 to step S26, the result of the personal authentication of the subject 1 is displayed on the display device 24 in step S26.

It should be noted that the face image of the subject 1 is an example of the face image of the subject according to the present disclosure. The face region image of the subject 1 generated from the scan data of the face image of the subject 1 is an example of the second face information according to the present disclosure and is an example of the second face region image.

### Example of ID Card Reading Selection Screen

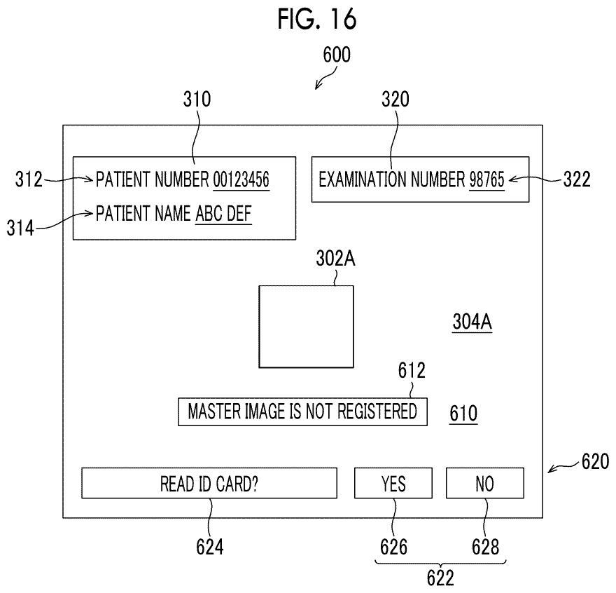

FIG. 16 is a schematic diagram of an ID card reading selection screen. An ID card reading selection screen 600 shown in FIG. 16 is displayed on the display device 24 in a case in which the master image of the subject 1 is not registered in the master image database 50 in step S100 of FIG. 15.

The ID card reading selection screen 600 includes the subject information display region 310 and the examination number display region 320 as in the master image display screen 300 shown in FIG. 7. The ID card reading selection screen 600 includes a master image display region 304A instead of the master image display region 304 shown in FIG. 7. The master image display region 304A includes a master image frame 302A instead of the master image 302.

The ID card reading selection screen 600 includes a notification information display region 610. The notification information display region 610 includes notification information 612 to which character information representing that the master image is not registered is applied. The operator who visually recognizes the notification information 612 can understand that the master image is not registered.

The ID card reading selection screen 600 includes a button display region 620. The button display region 620 includes one or more buttons 622 operated by the operator and processing information 624 representing the content of the processing.

FIG. 16 includes processing information 624 to which character information representing the reading of the ID card is applied, a button 626 operated in a case in which the processing is executed, and a button 628 operated in a case in which the processing is not executed.

In a case in which the operator who visually recognizes the ID card reading selection screen 600 determines that the master image of the subject 1 is not registered in the master image database 50, the operator can operate the button 626 operated in a case in which the processing is executed to perform the reading of the ID card of the subject 1.

### Example of Master Image Registration Screen

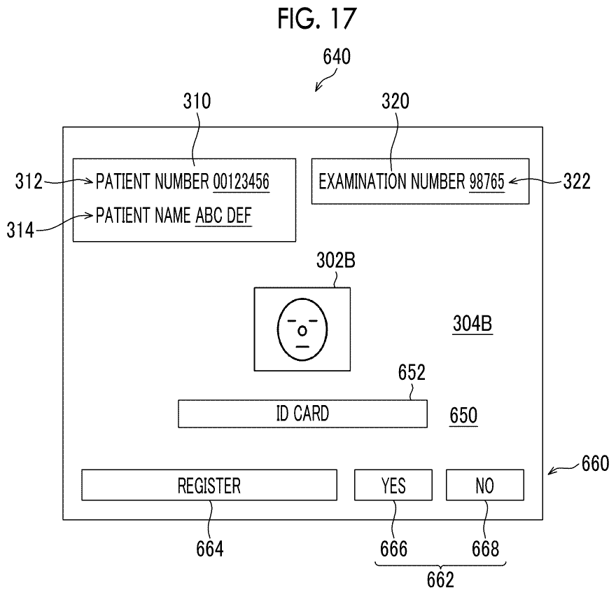

FIG. 17 is a schematic diagram of a master image registration screen. A master image registration screen 640 shown in FIG. 17 is displayed on the display device 24 in a case in which the master image 302B of the subject 1 based on the ID card is acquired in step S104 of FIG. 15.

The master image registration screen 640 includes the subject information display region 310 and the examination number display region 320 as in the master image display screen 300 shown in FIG. 7. The master image registration screen 640 includes a master image display region 304B instead of the master image display region 304 including the master image 302 acquired from the master image database 50 and the master image ID information 306. The master image display region 304B includes the master image 302B based on the scan data of the ID card.

The master image registration screen 640 includes a registration image information display region 650. The registration image information display region 650 includes registration image information 652 to which character information representing that the master image 302B is based on the scan data of the ID card is applied.

The master image registration screen 640 includes a button display region 660. The button display region 660 includes one or more buttons 662 operated by the operator and processing information 664 representing processing corresponding to the operation of the button 662. FIG. 17 shows processing information 664 to which character information representing processing of registering the master image 302B is applied, a button 666 operated in a case in which the processing is performed, and a button 668 operated in a case in which the processing is not performed.

The operator who visually recognizes the master image registration screen 640 can operate the button 666 to register the master image 302B of the subject 1 based on the scan data of the ID card in the master image database 50.

On the other hand, the operator can also operate the button 668 to prevent the registration of the master image 302B of the subject 1 based on the scan data of the ID card in the master image database 50.

### Actions and Effects of Second Embodiment

The authentication device 40A and the authentication method according to the second embodiment can obtain the following effects.

[1] In a case in which the master image 302 of the subject 1 is not registered in the master image database 50, the master image 302B of the subject 1 is generated based on the scan data of the face image included in the ID card of the subject 1. As a result, the personal authentication for the subject 1 for which the master image 302 is not registered in the master image database 50 is realized.

[2] The master image 302B of the subject 1 based on the scan data of the face image included in the ID card of the subject 1 is registered in the master image database 50. As a result, the subject 1 can use the master image 302 registered in the master image database 50.

### Authentication Device According to Third Embodiment

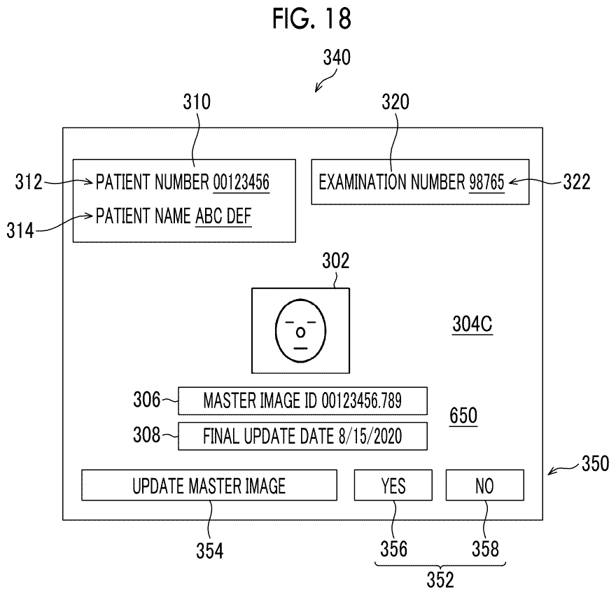

FIG. 18 is a schematic diagram of a master image update screen applied to the authentication device according to the third embodiment. The authentication device according to the third embodiment performs a notification for prompting re-registration in a case in which a predetermined period has elapsed from registration of the master image 302 acquired from the master image database 50.

A master image update screen 340 shown in FIG. 18 may be displayed on the display device 24 instead of the master image display screen 300 shown in FIG. 7 in step S12 shown in FIG. 6.

The master image update screen 340 includes the subject information display region 310 and the examination number display region 320 as in the master image display screen 300. The master image update screen 340 includes a master image display region 304C instead of the master image display region 304 shown in FIG. 7.

The master image display region 304C includes the master image 302 and the master image ID information 306 as in the master image display region 304, and further includes master image update information 308 representing a final update date. FIG. 18 shows an arbitrary date as the final update date.

The master image update screen 340 includes a button display region 350. The button display region 350 includes one or more buttons 352 operated by the operator and processing information 354 representing processing corresponding to the operation of the button 352. FIG. 18 shows processing information 354 to which character information representing processing of updating the master image 302 is applied, a button 356 operated in a case in which the processing is performed, and a button 358 operated in a case in which the processing is not performed.

The master image acquisition unit 122 shown in FIG. 4 and the master image acquisition unit 122A shown in FIG. 14 may acquire the final update date of the master image 302 from accessory information of the master image 302 acquired from the master image database 50, and generate the master image update screen 340 including the final update date of the acquired master image 302.

In a case in which a plurality of master images 302 are registered in the master image database 50 for the subject 1, a final update date of the latest master image 302 among the plurality of master images 302 may be included in the master image update screen 340, and the final update date of each of the plurality of master images 302 may be included in the master image update screen 340.

It should be noted that the master image for which the predetermined period has elapsed from registration is an example of the master face information for which the predetermined period has elapsed from registration according to the present disclosure.

### Actions and Effects of Third Embodiment

In the authentication device according to the third embodiment, the master image update information 308 representing the final update date of the master image 302 acquired from the master image database 50 is displayed on the master image update screen 340. As a result, the operator can determine whether or not to perform the personal authentication of the subject 1 using the acquired master image 302 based on the final update date of the master image of the subject 1, or to acquire another master image of the subject 1 and perform the personal authentication of the subject 1 using the other master image.

### Modification Example of Camera

The camera 30 fixedly disposed on the ceiling of the imaging room 17A shown in FIG. 1 and the like may be fixedly disposed on the wall of the imaging room 17A, or may be fixedly disposed using a support member such as a tripod erected on the floor.

A camera fixedly disposed above the subject 1 placed on the bed 14, such as a gantry camera built in the scan gantry unit 12, may be used instead of the camera 30 fixedly disposed on the ceiling of the imaging room 17A shown in FIG. 1 and the like, and the imaging of the subject 1 may be performed.

In a case in which the gantry camera is used to image the subject 1 placed on the bed 14, the subject 1 is imaged from an oblique upward direction. Processing of performing coordinate transformation or the like on the camera image of the subject 1 imaged from the oblique upward direction for the subject 1 may be performed to generate an image equivalent to the image in which the imaging of the subject is performed from directly above the subject 1.

### Modification Example of Subject Placed on Bed

A stretcher, a litter, or the like may be applied instead of the bed 14 shown in FIG. 1 and the like. A transport device that transports the subject 1 in a posture in which the subject 1 is lying down, such as the bed 14, the stretcher, and the litter, is provided with a subject support portion such as the top plate 14A of the bed 14 on which the subject 1 is supported while lying down. A posture of the subject 1 to be imaged is preferably a posture in which the face of the subject 1 faces the camera 30.

In a case in which the subject 1 is lying on the side instead of lying on the back, the face region image generation unit 126 shown in FIG. 4 generates a face region image of a side face of the subject 1. In a case in which the side face image of the subject 1 does not exist, it is difficult to match the face region image with the master image even for the same subject. Therefore, the camera image acquisition unit 124 may detect an orientation of the face of the subject 1 in the camera image of the subject 1, and may notify that the face of the subject 1 in the camera image faces the side.

In addition, the face region image generation unit 126 may convert the face region image of the subject 1 based on the camera image of the subject 1 in which the face faces the side into the face region image of the subject 1 in which the face faces the front.

The master image registration unit may register the face region image of the subject 1 based on the camera image of the subject 1 in which the face faces the side, in the master image database 50 as the side face image.

### Specific Example of Collation between Face Region Image of Subject and Master Image of Subject

The collation unit 128 shown in FIG. 4 may acquire various types of information such as a contour shape of the face, a distance interval between each part such as an eyebrow, an eye, a nose, a mouth, a cheek, and an ear, and a color of the skin from each of the face region image of the subject 1 and the master image of the subject 1, recognize a form of the human face as face data, and compare the face data of the two images with each other.

The collation unit 128 may calculate a similarity between the face data of the two images as a comparison result between the face data of the two images. The similarity between the face data of the two images may be calculated based on the similarity for each part.

The collation unit 128 may determine that the face region image of the subject 1 matches the master image of the subject 1 in a case in which the similarity exceeds a predetermined threshold value, and may determine that the face region image of the subject 1 does not match the master image of the subject 1 in a case in which the similarity is equal to or less than the predetermined threshold value.

### Application Example to Other Medical Image Capturing Apparatus

The application of the authentication device and the authentication method according to the embodiment is not limited to the medical image capturing apparatus being the X-ray CT apparatus. For example, the authentication device and the authentication method may be applied to a medical image capturing apparatus comprising a bed on which a subject is placed, such as an MRI apparatus and an XR apparatus. The MRI apparatus is an example of a magnetic resonance imaging apparatus according to the present disclosure, and the XR apparatus is an example of a medical image augmented reality apparatus according to the present disclosure.

### Hardware Configuration for Executing Each Processing

In the present embodiment, each processing is executed by any computer. Additionally, any computer may execute the processing through a processor, a program, or a combination thereof. Any computer may be a general-purpose computer, a computer for a specific purpose, a system such as a workstation, or other hardware elements capable of executing a program.

The processor may be configured by one or a plurality of pieces of hardware, and the type of hardware is not limited. For example, the processor may be configured by using hardware such as a central processing unit (CPU), a micro processing unit (MPU), a programmable logic device such as a field programmable gate array (FPGA), a dedicated circuit for executing specific processing such as an application specific integrated circuit (ASIC), a graphic processing unit (GPU), or a neural processing unit (NPU). In addition, the processor includes units or means that execute various types of processing in the present embodiment. Further, the type of hardware may be a combination of different types of hardware. In a case where a plurality of pieces of hardware are configured to execute one or a plurality of pieces of processing of a certain processor, the plurality of pieces of hardware may be present in devices physically separated from each other or may be present in the same device. Additionally, in any of the embodiments, the order of each processing by the processor is not limited to the above-described order and may be changed as appropriate. The hardware is configured by an electric circuit (circuitry) in which circuit elements, such as semiconductor elements, are combined, or the like.

Further, the present embodiment may be realized by hardware, software, firmware, microcode, or a combination thereof. Software, firmware, and microcode are composed of a program. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media existing in devices physically separated from each other. The program code or the code segment may represent any combination of procedures, functions, subprograms, routines, subroutines, modules, software packages, classes, or commands, data structures, or program statements. The program code or code segment may be connected to another code segment or a hardware circuit by transmitting and receiving information, data, an argument, a parameter, or a content of a memory.

### Application Example to Program and Program Product

The authentication method according to the embodiment may be configured as a program or a program product in which a processor or a computer including a processor realizes the functions of the respective steps.

The program or program product may be stored in a computer-readable medium that is a tangible non-transitory information storage medium, or may be provided through an information storage medium. Instead of the aspect in which the program is stored in the non-transitory computer-readable medium that is a tangible object and is provided, it is also possible to provide a program signal as a download service using an electric communication line.

The present disclosure is not limited to the above-described embodiments, and various modifications can be made without departing from the spirit of the technical idea of the present disclosure. In addition, each of the first to fourth embodiments can be combined as appropriate.

### EXPLANATION OF REFERENCES

1: subject
10: medical image capturing system
11: X-ray CT apparatus
12: scan gantry unit
12A: imaging space
14: bed
14A: top plate
16: operation unit
17A: imaging room
17B: operation room
20: examination control device
22: input device
24: display device
30: camera
40: authentication device
40A: authentication device
50: master image database
52: patient database
54: ID card reader
56: medical record database
62: X-ray source
64: rotating plate
65: opening portion
66: collimator
68: X-ray detector
70: data collection unit
80: rotating plate control unit
82: bed control unit
84: X-ray control unit
86: high-voltage generation unit
100: system control unit
102: examination information acquisition unit
104: image generation unit
106: storage device
108: automatic imaging position movement control unit
120: subject identification information acquisition unit
122: master image acquisition unit
122A: master image acquisition unit
124: camera image acquisition unit
126: face region image generation unit
128: collation unit
130: master image registration unit
130A: master image registration unit
132: subject information acquisition unit
202: processor
204: computer-readable medium
206: communication interface
208: input/output interface
210: bus
212: memory
214: storage
222: input device
224: display device
300: master image display screen
302: master image
302A: master image frame
302B: master image
304: master image display region
304A: master image display region
304B: master image display region
304C: master image display region
306: master image ID information
308: master image update information
310: subject information display region
312: patient number
314: patient name
320: examination number display region
322: examination number
330: button display region
332: OK button
340: master image update screen
350: button display region
352: button
354: processing information
356: button
358: button
400: camera image
402: subject image
404: horizontal center line
406: vertical center line
410: face region
412: center position
440: collation result display screen
440A: collation result display screen
440B: collation result display screen
442: image display region
442A: image display region
444: face region image
444A: face region image
450: collation result display region
450A: collation result display region
452: collation result
452A: collation result
460: button display region
460A: button display region
460B: button display region
462: button
462A: button
462B: button
464: processing information
464A: processing information
464B: processing information
466: button
466A: button
466B: button
468: button
468A: button
468B: button
500: authentication result display screen
500A: authentication result display screen
510: authentication result display region
510A: authentication result display region
512: authentication result
512A: authentication result
520: button display region
522: button
524: processing information
526: button
528: button
600: ID card reading selection screen
610: notification information display region
612: notification information
620: button display region
622: button
624: processing information
626: button
628: button
640: master image registration screen
650: registration image information display region
652: registration image information
660: button display region
662: button
664: processing information
666: button
668: button
S10 to S26, S100 to S104: each step of authentication method

What is claimed is:

1. An imaging support device comprising:
a processor; and
a memory that stores a program to be executed by the processor,
wherein the processor is configured to:
acquire a captured image of a subject who is lying down, the captured image being acquired by imaging the subject using an imaging apparatus that is fixedly disposed in an imaging room in which a medical image capturing apparatus is provided;
extract first face information of the subject from the captured image;
acquire master face information of the subject corresponding to identification information of the subject;
collate the first face information with the master face information; and
perform personal identification of the subject based on a collation result of the first face information and the master face information.

2. The imaging support device according to claim 1,
wherein the processor is configured to notify that the personal identification of the subject is successful in a case in which a similarity between the first face information and the master face information exceeds a predetermined value.

3. The imaging support device according to claim 1,
wherein the processor is configured to register the first face information as the master face information.

4. The imaging support device according to claim 3,
wherein the processor is configured to register the master face information in association with medical record information of the subject.

5. The imaging support device according to claim 1,
wherein the processor is configured to acquire the master face information for each subject from a master face information registration device in which the master face information for each subject associated with the identification information for each subject is registered.

6. The imaging support device according to claim 1,
wherein the processor is configured to notify information for prompting update of the master face information for which a predetermined period has elapsed from registration.

7. The imaging support device according to claim 1,
wherein the processor is configured to:
extract a face region of the subject from the captured image of the subject;
cut out the extracted face region from the captured image to generate a first face region image of the subject; and
apply the first face region image as the first face information.

8. The imaging support device according to claim 1,
wherein the processor is configured to:
acquire second face information of the subject from an ID card of the subject in a case in which the master face information of the subject is not registered; and
apply the acquired second face information as the master face information of the subject.

9. The imaging support device according to claim 8,
wherein the processor is configured to register the second face information as the master face information of the subject.

10. The imaging support device according to claim 8,
wherein the processor is configured to:
acquire scan data of a face image of the subject;
generate a second face region image of the subject from the scan data of the face image of the subject; and
apply the second face region image as the second face information.

11. A personal identification method performed by a computer that functions as an imaging support device including a processor and a memory that stores a program to be executed by the processor, the method comprising:
acquiring a captured image of a subject who is lying down, the captured image being acquired by imaging the subject using an imaging apparatus that is fixedly disposed in an imaging room in which a medical image capturing apparatus is provided,
extracting first face information of the subject from the captured image,
acquiring master face information of the subject corresponding to identification information of the subject,
collating the first face information with the master face information, and
performing personal identification of the subject based on a collation result of the first face information and the master face information.

12. A non-transitory, computer-readable tangible recording medium on which a program is recorded, the program for causing a computer that functions as an imaging support device including a processor and a memory that stores a program to be executed by the processor to implement functions of:
acquiring a captured image of a subject who is lying down, the captured image being acquired by imaging the subject using an imaging apparatus that is fixedly disposed in an imaging room in which a medical image capturing apparatus is provided;
extracting first face information of the subject from the captured image;
acquiring master face information of the subject corresponding to identification information of the subject;
collating the first face information with the master face information; and
performing personal identification of the subject based on a collation result of the first face information and the master face information.

13. A medical image capturing system comprising:
a medical image capturing apparatus that generates medical image capturing data of a subject;
an imaging apparatus that is fixedly disposed in an imaging room in which the medical image capturing apparatus is provided; and
the imaging support device according to claim 1 that performs personal identification of the subject.

14. The medical image capturing system according to claim 13,
wherein any one of a computed tomography imaging apparatus, a magnetic resonance imaging apparatus, or a medical image augmented reality apparatus is applied as the medical image capturing apparatus.

15. The medical image capturing system according to claim 13,
wherein the imaging apparatus is fixedly disposed on a ceiling of the imaging room, fixedly disposed on a wall of the imaging room, or fixedly disposed using a support member that is erected on a floor of the imaging room.

16. The medical image capturing system according to claim 13,
wherein the imaging apparatus is fixedly disposed on the medical image capturing apparatus.

17. The medical image capturing system according to claim 13,
wherein the medical image capturing apparatus includes a second processor and a second memory that stores a program to be executed by the second processor, and
the second processor is configured to:
detect a position of an imaging part of the subject in the captured image; and
perform movement control of a bed on which the subject is placed in the capturing of the medical image for the subject based on the detected position of the imaging part of the subject.
